(19) United States
(12) Patent Application Publication
(10) Pub. No.: US 2015/0307436 A1
(43) Pub. Date: Oct. 29, 2015

(54) METHOD FOR CONVERTING ODOR CONTAINING FATTY ACIDS TO DEODORIZED GLYCERIDES

(71) Applicant: The Procter & Gamble Company, Cincinnati, OH (US)

(72) Inventors: Jeffrey Charles CUMMINS, Loveland, OH (US); Manuel G. VENEGAS, West Chester, OH (US); Neil Thomas FAIRWEATHER, Liberty Township, OH (US); Stanley James WELLING, Hamilton, OH (US)

(21) Appl. No.: 14/684,493

(22) Filed: Apr. 13, 2015

Related U.S. Application Data
(60) Provisional application No. 61/983,530, filed on Apr. 24, 2014.

(51) Int. Cl. C07C 67/08

(52) U.S. Cl. CPC C07C 67/08

(57) ABSTRACT

A method for the production of deodorized glycerides from fatty acids containing odor bodies. The method comprises: (a) adding glycerine to the fatty acids to form a first mixture; (b) mixing the first mixture; (c) heating the first mixture at a temperature of from 120° C. to 250° C. to form a heated mixture; (d) removing water and odor bodies from the heated mixture; (e) optionally checking the amount of fatty acids remaining in the heated mixture; (f) optionally removing residual odor bodies from the heated mixture; and (g) recovering the deodorized glycerides.

### FIELD OF THE INVENTION

The present invention relates to the conversion of odor-containing fatty acids or odor-containing fatty acids with glycerides to deodorized glycerides with concomitant removal of odor bodies.

### BACKGROUND OF THE INVENTION

Palm-kernel and coconut oil constitute two of the most important seed oils. A substantial portion of fatty alcohols used for surfactant manufacture are produced through hydrogenation of fatty acid methyl esters that are derived from coconut and palm kernel oils. These fatty alcohols are used as a major component of soaps, detergents, and toiletries.

Fatty acid distillates (FAD) are by-products of the refining process used to prepare the crude oils for conversion to methyl esters. Without removal of the fatty acids, base catalysts used in methyl ester making will react with the fatty acids, forming soaps and resulting in lower yields. The FAD contains primarily fatty acids, but also contains glycerides and other minor components and impurities. For example, included in the impurities are odor-bodies resulting from the process of extracting the coconut oil from its source (coconuts), during which the coconut shells typically are burned. The burning creates phenols, furans, and other compounds of combustion giving a smoky, tar-like, burnt off-odor to the FAD. In order to prevent these odor bodies from affecting downstream operations (e.g., soap-making), the FADs containing these odor-causing compounds are removed along with the fatty acids as waste.

Accordingly, it would be desirable to prevent the yield loss caused by disposal of the FAD, yet still eliminate the odor-bodies from the downstream manufacturing process.

### SUMMARY OF THE INVENTION

The present invention eliminates the yield loss caused by disposal of the FAD, yet still eliminates the odor-bodies from the downstream manufacturing process. This is accomplished by converting the FAD into a useable material. The fatty acids present in the FAD stream are converted to glycerides, then the odor bodies are removed from the glycerides to form deodorized glycerides. The deodorized glycerides can be added back into the production stream or used in other downstream manufacturing processes (e.g., methyl esters, fatty alcohols).

### BRIEF DESCRIPTION OF THE DRAWINGS

FIG. 1 is a diagram showing the formation of glycerides from fatty acids according to the present invention. It should be understood that the particular mono- and di-glyceride positional isomers depicted do not limit the present invention to those isomers, but merely are representative of the positional isomers possible for monoglycerides (primary or secondary) and diglycerides (primary-primary or primary-secondary).

### DETAILED DESCRIPTION OF THE INVENTION

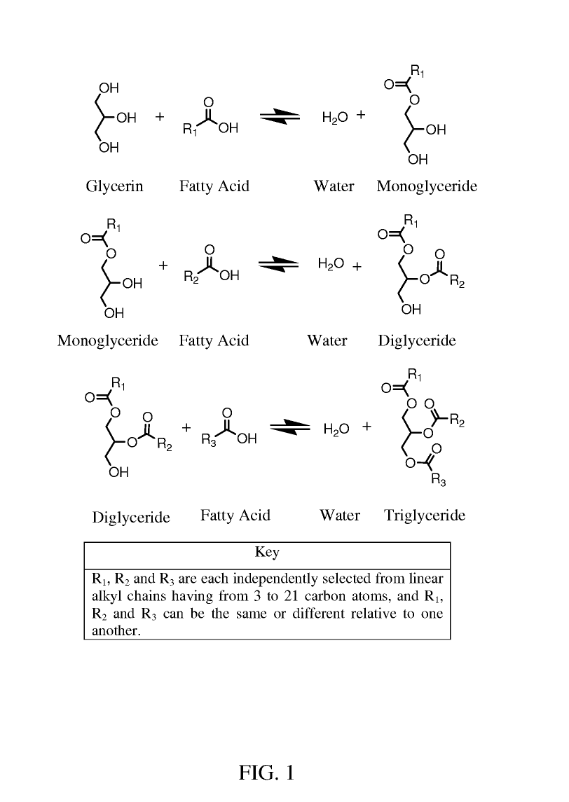

The present invention provides a method for the production of deodorized glycerides from fatty acids (or mixtures of fatty acids with glycerides) containing odor bodies. This process is shown in FIG. 1. As used herein, “fatty acid” includes (1) aliphatic monocarboxylic acids derived from or contained in esterified forms in an animal or vegetable fat, oil, or wax; (2) free fatty acids and/or glycerides (e.g., mono, di, tri) with odor bodies; and combinations thereof. The fatty acids herein typically have a carbon chain length of C₆-C₂₂, or C₈-C₁₈.

The fatty acid distillate (FAD) stream results from steam stripping of the crude vegetable oil to remove free fatty acids and odor compounds and typically contains about 70% fatty acids (C₈ through C₁₈ chain-length) and about 30% glycerides and odor compounds. In order to eliminate overall process yield loss, the present invention relates to converting these fatty acids back into glycerides for further processing (e.g., into methyl esters or fatty alcohols). This is accomplished by an esterification reaction of the FAD with glycerin, which converts the fatty acid first into a monoglyceride, which can react with another fatty acid making a diglyceride, which can react with a third fatty acid molecule making a triglyceride; water is formed in each of these reactions as a by-product. Each of these reactions is equilibrium based, resulting in a final mixture of glycerin, water, fatty acid, monoglycerides, diglycerides, and triglycerides.

Accordingly, the invention provides a method for the production of deodorized glycerides from fatty acids containing odor bodies, the method comprising:
(a) adding glycerine to the fatty acids to form a first mixture;
(b) mixing said first mixture;
(c) heating said first mixture at a temperature of from 120° C. to 250° C. to form a heated mixture.
(d) removing water and odor bodies from said heated mixture by a means selected from the group consisting of:
   (1) reducing the pressure of the heated mixture;
   (2) sparging the heated mixture with inert gas; and
   (3) combinations thereof;
(e) optionally checking the amount of fatty acids remaining in said heated mixture;
(f) optionally removing residual odor bodies from said heated mixture, wherein removing is selected from the group consisting of:
   (1) sparging the heated mixture with an inert gas;
   (2) steam stripping the heated mixture;
   (3) reducing the pressure of the heated mixture; and
   (4) combinations thereof;
(g) recovering the deodorized glycerides.

While use of the present invention is discussed mainly in terms of coconut oil, it should be understood that the method can be applied to any vegetable oil source as desired, such as palm kernel, palm or soybean. The present invention involves a method for the production of deodorized glycerides from fatty acids; in some cases, the fatty acid material will also comprise glycerides. In either case, the fatty acids (or fatty acids+glycerides) in the FAD are separated from the odor bodies contained therein to produce deodorized glycerides.

The method herein comprises adding glycerine to the fatty acids or (fatty acids+glycerides combination) containing odor bodies to form a first mixture. Such added glycerine can be selected from any suitable source, such as refined glycerine, crude glycerine, or combinations thereof. The first mixture is mixed using any appropriate mixing method and/or device as is known in the art. As used herein, “adding glycerine to the fatty acids” is broad enough to include the addition of glycerine to the aforementioned fatty acid+glyceride combination. Thus, unless otherwise indicated, “adding glycerine to the fatty acids” means adding glycerine to fatty acids that optionally may be in combination with glycerides.

It should be noted that the stoichiometric molar ratio of glycerine to fatty acid is 1:3 to produce a triglyceride product and water: 1 Glycerin+3 Fatty Acid→1 Triglyceride+3 H₂O (as shown in FIG. 1). The process of the present invention need not run stoichiometrically, however. The ratio of glycerine to FAD was determined empirically, targeting for optimization of fatty acid consumption at time and temperature. The preferred ratio of glycerin to FAD was determined to be about 25% by weight. This results in a mixture of mono-, di-, and triglycerides: 1 Glycerin+1, 2, or 3 Fatty Acids→1 Mono-, Di- or Triglyceride+1, 2, or 3 H₂O. This process is carried out without the use of catalyst.

After the mixing step, the mixture is then heated at a temperature of from 120° C. to 250° C., alternately from 190° C. to 210° C., or from 170° C. to 230° C., to form a heated mixture. As used herein, “heating at a temperature” means the temperature of the mixture being heated, rather than the temperature of the heat source.

Water and odor bodies are then removed from the heated mixture by any appropriate means; this step is preferably carried out concurrent with the heating step, wherein fatty acids react to form glycerides. The aforementioned heating and the subsequent process of removing water and odor bodies should be carried out such that the reaction rate is increased without substantially driving off the fatty acids. One skilled in the art will be able to balance the reaction rate with the drive-off of fatty acids to achieve an acceptable result. Means of removing water and odor bodies from the heated mixture during the reaction can include reducing the pressure of the heated mixture, sparging the heated mixture with inert gas (e.g., N₂, He, Ar), and/or combinations thereof.

At this point, one may optionally wish to check the amount of fatty acids remaining in the heated mixture, for example to compare with that level present in the mixture before heating. This can be accomplished by any suitable means known to one skilled in the art, such as GC, NMR, IR, titration, or other suitable methods. In one embodiment, this checking step comprises confirming that from 50% to 100%, or greater than 95%, of the fatty acids are no longer present in the heated mixture. If an undesired level of odor bodies still remains, these residual odor bodies are removed. Removing such residual odor bodies can be accomplished by any suitable means, such as sparging the heated mixtures with an inert gas (e.g., N₂, He, Ar), steam stripping the heated mixture, reducing the pressure of the heated mixture, and/or combinations thereof. In a particular embodiment, steam stripping is carried out for a time sufficient to reduce the level of odor bodies present in the glycerides to the desired level, as can be determined by one skilled in the art in view of this disclosure.

Once the desired level of odor body reduction is obtained, the resulting deodorized glycerides are recovered (i.e., when odor body reduction reaches an acceptable level, the glycerides are considered to be “deodorized”). At this point in the process, from 2 to 12 hours, or from 5 to 7 hours, have typically elapsed. The desired level of odor body reduction will depend upon various factors, such as the intended use of the deodorized glycerides. One skilled in the art will be able to determine the desired level of odor body reduction in view of this disclosure.

### EXAMPLES

### Examples 1-6

### General Reaction Set-Up and Materials

A 3-neck round bottom flask or multi-port reaction vessel was utilized with a temperature controlled heating mantle and stiffing. One neck was used for a nitrogen addition tube, one neck was used to vent and/or apply vacuum to the nitrogen stream through a condenser and collection vessel, and the final neck was used for placement of a thermocouple to monitor and control temperature. Fatty acid distillate (FAD) and glycerin were both sourced from P&G Chemicals.

### Example 1

800 g of FAD and 200 g of glycerin were added to the flask followed by warming to ˜200° C. During heating, a nitrogen sweep (˜50 mL/s) was started. The reaction was held under these conditions for a total of 6 hours with water and odor bodies distilled and collected during the reaction. Samples were removed from the reaction flask at the times shown in Table 1 below and analyzed by GC with the % remaining fatty acid reported.

TABLE 1
Summary of GC data

| Sample | Time (Hrs) | Total COOH (%) |
|---|---|---|
| FAD | 0 | 63.88 |
| 1 | 1 | 16.3 |
| 2 | 2 | 9.25 |
| 3 | 3 | 5.63 |
| 4 | 4 | 3.93 |
| 5 | 5 | 2.64 |
| 6 | 6 | 1.85 |

### Example 2

200 g of FAD and 50 g of glycerin were added to the flask followed by warming to ˜175° C. During heating, a nitrogen sweep (˜25 mL/s) was started. The reaction was held under these conditions for a total of 10 hours with water and odor bodies distilled and collected during the reaction. Samples were removed from the reaction flask at the times shown below in Table 2 and analyzed by GC with the % remaining fatty acid reported.

TABLE 2
Summary of GC data

| Sample | Time (Hrs) | Total COOH (%) |
|---|---|---|
| FAD | 0 | 63.88 |
| 1 | 2 | 20.7 |
| 2 | 4 | 9.62 |
| 3 | 6 | 5.47 |
| 4 | 8 | 3.28 |
| 5 | 10 | 1.97 |

### Example 3

6000 g of FAD and 1500 g of glycerin were added to the reaction vessel followed by warming to ˜200° C. During heating, a nitrogen sweep (˜120 mL/s) was started. The reaction was held under these conditions for a total of 7 hours with water and odor bodies distilled and collected during the reaction. Samples were removed from the reaction flask at the times shown below (Table 3) and analyzed by GC with the % remaining fatty acid reported.

TABLE 3
Summary of GC data

| Sample | Time (Hrs) | Total COOH (%) |
|---|---|---|
| FAD | 0 | 73.92 |
| 1 | 1 | 31.4 |
| 2 | 4 | 4.07 |
| 3 | 6 | 1.22 |
| 4 | 7 | 0.45 |

### Example 4

6000 g of FAD and 1500 g of glycerin were added to the reaction vessel followed by warming to ˜200° C. During heating, a vacuum (˜300 Torr) was started. The reaction was held under these conditions for a total of 7 hours with water and odor bodies distilled and collected during the reaction. Samples were removed from the reaction flask at the times shown below (Table 4) and analyzed by GC with the % remaining fatty acid reported.

TABLE 4
Summary of GC data

| Sample | Time (Hrs) | Total COOH (%) |
|---|---|---|
| FAD | 0 | 73.92 |
| 1 | 1 | 30.32 |
| 2 | 4 | 5.27 |
| 3 | 6 | 2.31 |
| 4 | 7 | 1.48 |

### Example 5

400 g of FAD and 100 g of glycerin were added to the reaction vessel followed by warming to ˜210° C. During heating, a nitrogen sweep of (˜1 mL/min) was started. The reaction was held under these conditions for a total of 5 hours with water and odor bodies distilled and collected during the reaction. Samples were removed from the reaction flask at the times shown below (Table 5) and analyzed by GC with the % remaining fatty acid reported.

TABLE 5
Summary of GC data

| Sample | Time (Hrs) | Total COOH (%) |
|---|---|---|
| FAD | 0 | 66.85 |
| 1 | 1 | 9.72 |
| 2 | 2 | 4.06 |
| 3 | 3 | 1.47 |
| 4 | 4 | 0.19 |
| 5 | 5 | 0 |

### Example 6

6000 g of FAD and 1500 g of glycerin were added to the reaction vessel followed by warming to ˜200° C. During heating, a vacuum (˜300 Torr) was started. The reaction was held under these conditions for a total of 6 hours with water and odor bodies distilled and collected during the reaction. To further reduce the level of fatty acids and odor compounds, the reactants were then steam stripped by adding 500 mL of water to the batch over a period of 1 hour. This was repeated for a total of 5 times. Samples were removed from the reaction flask at the times shown below (Table 6) after each steam stripping and analyzed by GC with the % remaining fatty acid reported.

TABLE 6
Summary of GC data

| Sample | Time (Hrs) | Total COOH (%) |
|---|---|---|
| FAD | 0 | 73.92 |
| 1 | 1 | 27.11 |
| 2 | 3 | 5.63 |
| 3 | 6 | 1.63 |
| Steam Strip 1 | — | 0.90 |
| Steam Strip 2 | — | 0.85 |
| Steam Strip 3 | — | 0.55 |
| Steam Strip 4 | — | 0.09 |
| Steam Strip 5 | — | 0 |

### Example 7

General reaction set-up and materials: A 100 gallon glass-lined Pfaudler reactor was used for the reaction containing a center agitator as well as a condenser and a dip-tube through the reactor head down to the bottom of the reactor. Steam ejectors are used to pull vacuum on the system and the reactor heated by a hot oil jacket.

Procedure: 185 kg of FAD was added to the reactor and heated to ˜190° C. under ˜350 mmHg vacuum. 46 kg of glycerin was added to the reactor once at temperature. The reaction was held under these conditions for a total of 12 hours with water and odor bodies distilled and collected during the reaction. Samples were removed from the reactor every hour and analyzed by GC with results shown in the table below (Table 7) with the % remaining fatty acid reported.

TABLE 7
Summary of GC data before steam stripping

| Sample | Time (Hrs) | Total COOH (%) |
|---|---|---|
| FAD | 0 | 73.46 |
| 1 | 1 | 56.43 |
| 2 | 2 | 11.75 |
| 3 | 3 | 7.00 |
| 4 | 4 | 2.65 |
| 5 | 5 | 1.91 |
| 6 | 6 | 1.43 |
| 7 | 7 | 1.11 |
| 8 | 8 | 0.90 |
| 9 | 9 | 0.75 |
| 10 | 10 | 0.65 |
| 11 | 11 | 0.56 |
| 12 | 12 | 0.57 |

After this time, the pressure was reduced to ˜6 mmHg Water was then added to the reactor through the dip-tube slowly in 10 lb increments to generate steam in situ. Samples from the reactor were collected after 60, 120, and 180 lbs. of water in total were added to the system, and analyzed by GC with the % remaining fatty acid reported in the table below (Table 8).

TABLE 8
Summary of GC data after steam stripping

| Total Water Added (lbs) | Total COOH (%) |
|---|---|
| 60 | 0.30 |
| 120 | 0.31 |
| 180 | 0.29 |

### Example 8

### Sensory Evaluation

A ten-person panel qualitatively evaluated a variety of samples via olfactory analysis. The samples included both crude oil (prior to FAD removal) and deodorized glycerides (processed using nitrogen sweep, reduced pressure, and/or steam stripping according to the method herein). The panel concluded that the deodorized glyceride samples had a better odor profile than did the crude oil samples.

### Example 9

### Further Processing of Deodorized Glycerides

Using procedures available in the chemical literature, deodorized glycerides prepared as outlined above were converted to methyl esters using sodium methoxide and methanol. The obtained methyl esters were hydrogenated using a copper chromite catalyst to form the desired fatty alcohols, followed by conversion of the alcohols to alkyl sulfates using chlorosulfonic acid and sodium hydroxide. The alkyl sulfates were evaluated by a qualified panel and judged to have acceptable odor profiles.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for the production of deodorized glycerides from fatty acids containing odor bodies, said method comprising:
(a) adding glycerine to the fatty acids to form a first mixture;
(b) mixing said first mixture;
(c) heating said first mixture at a temperature of from 120° C. to 250° C. to form a heated mixture.
(d) removing water and odor bodies from said heated mixture by a means selected from the group consisting of:
(1) reducing the pressure of the heated mixture;
(2) sparging the heated mixture with inert gas; and
(3) combinations thereof;
(e) optionally checking the amount of fatty acids remaining in said heated mixture;
(f) optionally removing residual odor bodies from said heated mixture, wherein removing is selected from the group consisting of:
(1) sparging the heated mixture with an inert gas;
(2) steam stripping the heated mixture;
(3) reducing the pressure of the heated mixture; and
(4) combinations thereof;
(g) recovering the deodorized glycerides.

2. The method of claim 1, wherein heating said first mixture in step (c) comprises heating at a temperature of from 190° C. to 210° C.

3. The method of claim 1, wherein heating said first mixture in step (c) comprises heating at a temperature of from 170° C. to 230° C.

4. The method of claim 1, wherein the level of glycerine added in step (a) is an amount in excess of a stoichiometric amount.

5. The method of claim 1, wherein the level of glycerine added in step (a) is an amount less than or equal to a stoichiometric amount.

6. The method of claim 1, wherein said inert gas is selected from Nitrogen, Helium, and Argon.

7. The method of claim 1, wherein steps (c) and (d) increase the reaction rate without substantially driving off the fatty acids.

8. The method of claim 1, wherein checking in step (e) comprises confirming that from 50% to 100% of the fatty acids are no longer present in the heated mixture.

9. The method of claim 11, wherein checking in step (e) comprises confirming that greater than 95% of the fatty acids are no longer present in the heated mixture.

10. The method of claim 1, wherein said glycerine added in step (a) is selected from refined glycerine, crude glycerine, and combinations thereof.

11. The method of claim 1, wherein steps (a)-(g) are performed in 2 to 12 hours.

12. The method of claim 1, wherein steps (a)-(g) are performed in 5 to 7 hours.

13. The method of claim 1, wherein said steam stripping of step (f) is carried out for a time sufficient to reduce the level of odor bodies present in the glycerides to the desired level.
